(19) United States
(12) Patent Application Publication
(10) Pub. No.: US 2016/0199290 A1
(43) Pub. Date: Jul. 14, 2016

(54) CONCENTRATED MASCARA AND EYELINER SYSTEMS FOR DEVELOPMENT OF NEW COSMETIC PRODUCTS

(71) Applicant: Kobo Products, Inc., South Plainfield, NJ (US)

(72) Inventors: Yun Shao, Belle Mead, NJ (US); Edward Bartholomey, Rosedale, MD (US)

(73) Assignee: KOBO PRODUCTS, INC., South Plainfield, NJ (US)

(21) Appl. No.: 14/410,505

(22) PCT Filed: Jun. 24, 2013
(86) PCT No.: PCT/US13/47440

§ 371 Date: Dec. 22, 2014

Related U.S. Application Data
(60) Provisional application No. 61/662,990, filed on Jun. 22, 2012.

(51) Int. Cl. A61K 8/92; A61K 8/81; A61K 8/19; A61K 8/88; A61K 8/64; A61Q 1/10; A61K 8/02; A61K 8/04

(52) U.S. Cl. CPC A61K 8/922; A61K 8/0241; A61K 8/8111; A61K 8/04; A61K 8/88; A61K 8/64; A61Q 1/10; A61K 8/19; A61K 2800/412; A61K 2800/524; A61K 2800/48; A61K 2800/594; A61K 2800/43

(57) ABSTRACT

This application discloses an aqueous dispersion of waxes, polymers, pigments and other functional additives, which can be used as a concentrate of an eye make-up composition. The dispersion contains about 70% solids and can be diluted at room temperature and adjusted with additional ingredients if desired, which enables commercial cosmetic manufacturers to develop and manufacture new formulations with the minimal effort and cost.

### CROSS REFERENCE TO RELATED APPLICATIONS

This application claims priority under 35 U.S.C. §119(e) to U.S. Provisional Application Ser. No. 61/662,990, filed Jun. 22, 2012, which is incorporated by reference in its entirety.

### FIELD OF THE INVENTION

This invention relates to an aqueous dispersion of waxes, polymers, pigments and other functional additives, and its use as a concentrate of eye make-up compositions.

### BACKGROUND OF THE INVENTION

Mascara formulation is usually a viscous emulsion with ingredients such as waxes, emollients, pigments and film formers. In the production process, waxes, fatty acids and emollients are mixed and heat to 80-90° C. to melt and dissolve the waxes. Pigments and emulsifier then are added and dispersed in this oily mixture. Hydrophilic thickeners like gums or synthetic polymers are dispersed in hot water. Some of them require neutralization. This second phase is heated and combined with the oily phase to form the emulsion.

Mascara and eyeliner formulations incorporate anionic soaps and non-ionic emulsifiers, such as glycerides and fatty alcohols. Their primary function is to emulsify waxes to sub-micron size and stabilize the Particles.

However, emulsifiers can reduce the desired water-proofness because they cause films on lash to dissolve due to their hydrophilic nature. Some emulsifier can also be a source of skin and eye irritation. Many of the common emulsifiers for mascaras and eyeliners have regulatory issues and remain as questionable for the future, especially in Europe. Non ionic systems are preferred by many cosmetic companies. Less emulsifier provides better performance.

The conventional manufacturing necessitate the heating and precise cooling, two sets of heating tanks and mixers. This not only incurs energy cost but also extends the production time because some processes cannot proceed until a certain temperature is reached.

Moreover, processing, weighing, quality control and inspection of raw materials for a typical conventional process add the cost significantly. The quality assurance involves many aspects. In the current trend of cost-cutting, in order to remain competitive in the marketplace, it is desired to have a concentrate in the formulation process as a single component that contains all keep ingredients at an optimized ratio.

Aqueous mascaras are wax-in-water emulsions that produce sub-micron Particles of waxes. Waxes are known to be melted and then emulsified to form wax-in-water emulsions which may then be used to produce both mascara and eyeliner products. Waxes are seldom added directly to an aqueous phase. EP394078 disclosed a cosmetic composition for hair treatment containing a microdispersion of wax in water using ionic or non-ionic emulsifier. U.S. Pat. No. 5,849,278 disclosed an eye makeup composition comprises an aqueous dispersion of wax Particles, at least one water-soluble film-forming polymer and pigments, the said dispersion being a microdispersion of colloidal wax Particles. The inventor claim that the addition of water-soluble film-forming polymers help the formula to exhibit remarkable qualities of regular and smooth deposits on the eyelashes. US20110250148 (A1) disclosed water dispersible wax powders for cold process emulsification. Water soluble polymers such as sodium polyacrylate are used to form a composite with waxes and help to dispersion the wax at room temperature. However, the water-soluble polymer can lead to a poor water resistance of the final eye make-up composition.

### SUMMARY OF THE INVENTION

It is an object of this invention to provide an aqueous dispersion of waxes, polymers, pigments and other functional additives, which can be used as a concentrate of an eye make-up composition.

It was discovered, in a surprising manner, that when the all Particulate ingredients such as pigments, functional filler are incorporated to a solid loading of 70%, no additional emulsifier is needed to maintain the stability. Both the concentrate and dilutions of this technology to form mascaras & eyeliners have improved water-resistance over conventional products.

In one aspect, the present invention provides a dispersion composition comprising one or more waxes, one or more water-insoluble film-forming polymers, one or more pigments and other functional additives, wherein said dispersion has a viscosity in the range of about 150,000 cPs. to about 800,000 cPs. at about 25° C. and comprises at least about 60% of solids by weight.

In one embodiment of this aspect, the dispersion composition is an aqueous dispersion, optionally containing an organic solvent.

It is another object of this invention to provide a process for manufacturing the said dispersion using a cold process or a low heat process when the temperature is below the melting point of the wax.

In another aspect, the present invention provides a method of making a dispersion composition as described herein, the method comprising:

a) adjusting a polymer emulsion or dispersion with a base to a desired pH;

b) adding one or more waxes to the polymer emulsion or dispersion under agitation;

c) adding one or more functional additives to the mixture under agitation; and

d) adding one or more preservatives under agitation.

In another aspect, the present invention provides a method of making an eye composition, comprising:

a) mixing one or more diluents with one or more preservatives to form a mixture;

b) adding a dispersion composition according to any embodiments described herein and mixing till uniform; and

c) filling with mixture obtained in b) with one or more filler materials.

In preferred embodiments, no heating, cooling or homogenization is applied in the manufacturing processes.

It is still another object of this invention to provide a method for making final eye makeup formulation from the above concentrated dispersion. Thus, in another aspect, the present invention provides rub-resistant and water-resistant mascara incorporating a dispersion composition according to any embodiment described herein.

These and other aspects of the present invention will be better appreciated by reference to the following detailed description and claims.

### DETAILED DESCRIPTION OF THE INVENTION

This invention provides an aqueous dispersion of waxes, polymers, pigments and other functional additives, which can be used as a concentrate of an eye make-up composition.

In one aspect, the present invention provides a dispersion composition comprising one or more waxes, one or more water-insoluble film-forming polymers, one or more pigments and other functional additives, wherein said dispersion has a viscosity in the range of about 150,000 cPs. to about 800,000 cPs. at about 25° C. and comprises at least about 60% of solids by weight.

In one embodiment of this aspect, the dispersion composition is an aqueous dispersion, optionally containing an organic solvent, such as alchol (e.g., EtOH, isopropanol, or the like), or glycols, such as Caprylyl Glycol, Hexylene Glycol, or the like.

In another embodiment of this aspect, the one or more waxes comprise a plurality of wax particles.

In another embodiment of this aspect, the one or more waxes have particle sizes in the range of about 0.5 microns to about 50 microns (0.5-50 μm) in diameter.

In another embodiment of this aspect, the one or more waxes have particle sizes in the range of about 2 microns to about 10 microns (2-10 μm) in diameter.

In another embodiment of this aspect, the one or more waxes are independently selected from natural waxes.

In another embodiment of this aspect, the natural waxes comprise Carnauba wax, Candellila wax, Alfa wax, and Beeswax.

In another embodiment of this aspect, the one or more waxes are independently selected from synthetic waxes.

In another embodiment of this aspect, the synthetic waxes comprise polyethylene and microcrystalline waxes.

In another embodiment of this aspect, the one or more waxes comprise both synthetic and natural waxes.

In another embodiment of this aspect, the one or more waxes are present in the form of particles comprising a composite of synthetic and natural waxes.

In another embodiment of this aspect, the particles of said one or more waxes are in the range from about 1% to about 30% by weight of the total composition.

In another embodiment of this aspect, the one or more water-insoluble film-forming polymers are emulsion of synthetic polymer emulsion or dispersion.

In another embodiment of this aspect, the one or more water-insoluble film-forming polymers are in the range of about 1% to about 30% by weight of the total composition.

In another embodiment of this aspect, the one or more water-insoluble film-forming polymers are independently selected from acrylate polymers or copolymers, polyurethanes, polyvinyl acetate and copolymers, styrene-butadiene copolymers, vinyl acetate-ethylene copolymers.

In another embodiment of this aspect, the one or more pigments are in the range of from about 2% to about 50% by weight of the total composition.

In another embodiment of this aspect, the one or more additive fillers comprise particulate materials.

In another embodiment of this aspect, the additive fillers are in a form selected from microspheres and fibers.

In another embodiment of this aspect, the microsphere has a size of about 1 μm to about 50 microns (1-50 μm) in diameter.

In another embodiment of this aspect, the microsphere is made from a mineral or polymeric material.

In another embodiment of this aspect, the fibers have a length in the range from about 0.1 mm to about 10 mm (0.1-10 mm) and a thickness in the range from about 0.5 denier to about 10 denier (0.5-10 D).

In another embodiment of this aspect, the fibers are made from a synthetic polymer or natural fibers.

In another embodiment of this aspect, the synthetic polymer is selected from Nylons.

In another embodiment of this aspect, the natural fiber is silk.

In some embodiments, the dispersion of the present invention can be used as is, or diluted with water and some aqueous gel to make regular mascara, or can be diluted much further to make an eye liner composition.

In some embodiments, the composition contains a latex (aqueous emulsion or dispersion of polymer) at 5-50% by solids. Suitable latex includes acrylic latex, natural latex, any dispersion or emulsion of synthetic polymer. Examples of acrylic latex are Daitosol 5000SJ, Daitosol 5000AD from Daito Kasei Kogyo. The latex may need to be neutralized before mixing with other ingredient.

In some embodiments, the composition contains micronized wax powders. Suitable waxes are natural waxes such as Carnauba wax, Beeswax, Ozokerite or synthetic waxes such as polyethylene, microcrystalline wax, etc. The wax can also be a mixture of natural and synthetic waxes. The wax powder can have any shape, irregular, granular or spherical. The mean sizes of these powders are in the range of 0.5-50 microns, but preferably, 2-10 microns.

In some embodiments, the composition contains pigments such as iron oxides, ultramarines, titanium dioxide, and Black No. 2 (carbon black). The pigments can also be water dispersible lakes of organic dyes. It is preferred that the pigment be pre-dispersed in water and milled to achieve full color strength.

In some embodiments, the composition may also contain other functional filler to improve its performance. Microspheres with a mean size of 3-50 microns, preferably, 5-20 microns, can be used to provide volumizing effect. The micropsheres can be made from synthetic or natural polymers such as PMMA (SUNPMMA-S from Sunjin), Cellulo Bead from Daito Kasei, or, from inorganic substrate such as silica (MSS-500/20N, MSS-500/N from Kobo).

In some embodiments, the fiber can be added to the dispersion to provide lengthening effect. Suitable fibers are natural fiber such as Cell-u-Lash from Kobo and synthetic fiber such as Nylon Cut Fiber 3D (Kobo/Jigen International)

In some embodiments, the aqueous thickeners can be used to stabilize the dispersion. The thickeners can be natural gum like xanthan gum or synthetic clay, such as bentonite.

The production process for enabling cosmetic manufacturers (see, e.g., Examples 6-13) involves only mixing at room temperature with a Cowles Mixer or the equivalent. No heating or homogenization is needed.

In another aspect, the present invention provides a method of making a dispersion composition as described herein, the method comprising:

a) adjusting a polymer emulsion or dispersion with a base to a desired pH;

b) adding one or more waxes to the polymer emulsion or dispersion under agitation;

c) adding one or more functional additives to the mixture under agitation; and

d) adding one or more preservatives under agitation.

In a preferred embodiment of this aspect, no heating, cooling, or homogenization is applied.

In another aspect, the present invention provides a method of making an eye composition, comprising:

a) mixing one or more diluents with one or more preservatives to form a mixture;

b) adding a dispersion composition according to any embodiments described herein and mixing till uniform; and

c) filling with mixture obtained in b) with one or more filler materials.

In one embodiment of this aspect, the diluents are selected from water, oil, silicone, and hydrocarbon fluids.

In another embodiment of this aspect, the method further includes adding a thickener to stabilize the final product.

In another embodiment of this aspect, the method further includes adding one or more additional ingredients to make performance claims.

In a preferred embodiment of this aspect, no heating, cooling or homogenization is applied.

In another embodiment, the present invention provides a rub-resistant mascara incorporating a dispersion composition according to any embodiment described herein.

In another embodiment, the present invention provides a water-resistant mascara incorporating the dispersion composition according to any embodiment described herein.

In another embodiment, the present invention provides a mascara incorporating the dispersion composition of according to any embodiment described herein, which is both water resistant and rub resistant. In another embodiment, the water and rub-resistant mascara has a dark color as measured by an L value in the range of 9 to 11.

The present invention is additionally described by the way of the following illustrative, non-limiting examples that provides a better understanding of the present invention and of its advantage.

### EXAMPLES

### Example 1

### Lengthening Mascara Concentrate [Cold Process]

| | % W/W |
|---|---|
| Part A | |
| Triethanolamine 99% | 0.29 |
| Daitosol 5000SJ | 24.03 |
| (Acrylates/Ethylhexyl Acrylate Copolymer, Water) | |
| Deionized Water | 2.86 |
| Dow Corning ® Antifoam C | 1.00 |
| Part B | |
| Microcare ® 310 (polyethylene, Carnauba wax) | 4.75 |
| Micropoly ® 250S (polyethylene,) | 9.00 |
| Part C | |
| SUN PMMA-S (PMMA from Sunjin) | 18.25 |
| NFCB-10D-2T (Nylon-12 fiber) | 1.75 |
| Part D | |
| W60BBNFAP-O (from Kobo) | 34.60 |
| (60% Black iron oxide dispersion in water) | |
| Part E | |
| Xanthan Gum | 0.35 |
| Optiphen ® (Caprylyl Glycol, Phenoxyethanol) | 0.65 |
| Symdiol ® 68 (1,2-Hexanediol, Caprylyl Glycol) | 0.60 |
| Part F | |
| Ethyl Alcohol SDA 40B - 190 Proof | 1.72 |
| Sorbic Acid NF | 0.15 |
| Total: | 100.00 |

### Procedure

1. For Part A combine the TEA and Daitosol. Mix together gently. Adjust the pH level to 8.00-8.20 using TEA. Add the Water and Antifoam with very low stirring for 5 minutes.

2. Add the waxes in Phase B to Part A. Disperse with the Cowles Mixer at low speed for 20 minutes.
3. Add the NFCB-10D-2T in Part C and stir with the Cowles Mixer at low speed for 20 minutes.

The volume of the batch will rise due to the nylon.
4. Add the PMMA and stir with the Cowles Mixer at low speed for 20 minutes.
5. Add PART D pigment slurry to ABC. The viscosity will become thinner. Disperse with the Cowles Mixer at low speed for 20 minutes.
6. Slurry together the ingredients of PART F and add to Phase ABCD. Disperse very slowly with the Cowles Mixer at low speed for 20 minutes. Continue stirring until the air is reduced to a minimum.
7. Add the sorbic acid to the ethanol in PART E and stir together for 5 minutes. Add to the batch ABCDE. Disperse for 15 minutes.

### Example 2

### Thickening Mascara Concentrate [Cold Process]

| | % W/W |
|---|---|
| Part A | |
| Triethanolamine 99% | 0.29 |
| Daitosol 5000SJ | 24.03 |
| (Acrylates/Ethylhexyl Acrylate Copolymer, Water) | |
| Deionized Water | 0.21 |
| Dow Corning ® Antifoam C | 1.00 |
| Part B | |
| Microcare ® 310 (polyethylene, Carnauba wax) | 4.75 |
| Micropoly ® 250S (polyethylene) | 9.00 |
| Part C | |
| SUN PMMA-S (PMMA, Sunjin) | 18.25 |
| MSS-500/N (Silica, Kobo) | 5.00 |
| Part D | |
| W60BBNFAP-O (from Kobo) | 34.00 |
| (Black iron oxide dispersion in water) | |
| Part E | |
| Xanthan Gum | 0.35 |
| Optiphen ® (Caprylyl Glycol, Phenoxyethanol) | 0.65 |
| Symdiol ® 68 (1,2-Hexanediol, Caprylyl Glycol) | 0.60 |
| Part F | |
| Ethyl Alcohol SDA 40B - 190 Proof | 1.72 |
| Sorbic Acid NF | 0.15 |
| Total: | 100.00 |

### Procedure

1. For Part A combine the TEA and Daitosol. Mix together gently. Add the Water and Antifoam with very low stirring for 5 minutes. Adjust the pH level to 8.00-8.20 using TEA.
2. Add the waxes in Phase B to Part A. Disperse with the Cowles Mixer at low speed for 20 minutes.
3. Add the Silica beads MSS-500/N in Part C and stir with the Cowles Mixer at low speed for 20 minutes.
4. Add the PMMA and stir with the Cowles Mixer at low speed for 20 minutes.
5. Add PART D pigment slurry to ABC. The viscosity will become thinner. Disperse with the Cowles Mixer at low speed for 20 minutes.
6. Slurry together the ingredients of PART E and add to Phase ABCD. Disperse very slowly with the Cowles Mixer at low speed for 20 minutes. Continue stirring until the air is reduced to a minimum.
7. Add the sorbic acid to the ethanol in PART F and stir together for 5 minutes. Add to the batch ABCDE. Disperse for 15 minutes.

### Example 3

### Eyeliner Concentrate [Cold Process]

| | % W/W: |
|---|---|
| Part A | |
| Triethanolamine 99% | 0.28 |
| Daitosol 5000SJ | 30.24 |
| (Acrylates/Ethylhexyl Acrylate Copolymer, Water) | |
| Deionized Water | 1.73 |
| Dow Coming ® Antifoam C | 1.00 |
| Part B | |
| Microcare ® 310 (polyethylene, Carnauba wax) | 2.50 |
| Micropoly ® 250S (polyethylene) | 5.00 |
| Part C | |
| SUN PMMA-S (PMMA, Sunjin) | 18.25 |
| Part D | |
| W60BBNFAP-O (from Kobo) | 28.13 |
| (60% Black iron oxide dispersion in water) | |
| WBG20CB (from Kobo) | 9.60 |
| (20% Black No. 2 dispersion in water) | |
| Part E | |
| Xanthan Gum | 0.15 |
| Optiphen ® (Caprylyl Glycol, Phenoxyethanol) | 0.65 |
| Symdiol ® 68 (1,2-Hexanediol, Caprylyl Glycol) | 0.60 |
| Part F | |
| Ethyl Alcohol SDA 40B - 190 Proof | 1.72 |
| Sorbic Acid NF | 0.15 |
| Total: | 100.00 |

### Procedure

1. For Part A combine the TEA and Daitosol. Mix together gently. Add the Water and Antifoam with very low stirring for 5 minutes. Adjust the pH level to 8.00-8.20 using TEA.
2. Add the waxes in Phase B to Part A. Disperse with the Cowles Mixer at low speed for 20 minutes.
3. Add the PMMA and stir with the Cowles Mixer at low speed for 20 minutes.
4. Add PART D both pigment slurries to ABC. The viscosity will become thinner. Disperse with the Cowles Mixer at low speed for 20 minutes.
5. Slurry together the ingredients of PART F and add to Phase ABCD. Disperse very slowly with the Cowles Mixer at low speed for 20 minutes. Continue stirring until the air is reduced to a minimum.
6. Add the sorbic acid to the ethanol in PART E and stir together for 5 minutes. Add to the batch ABCDE. Disperse for 15 minutes.

### Example 4

### Lengthening Mascara Concentrate (Low-Heat Process)

| | % W/W |
|---|---|
| PART A | |
| Triethanolamine 99% | 0.29 |
| Daitosol 5000SJ | 24.03 |
| Deionized Water | 2.86 |
| Antifoam C | 1.00 |
| PART B | |
| Cetearyl Alcohol and Ceteareth-20 | 8.85 |
| Micropoly ® 250S (polyethylene) | 5.00 |
| PART C | |
| NFCB-10D-2T | 1.75 |
| PART D | |
| W60BBNFAP-O (Kobo Products) | 34.50 |
| PART E | |
| SUN PMMA-S | 18.25 |
| PART F | |
| Xanthan Gum | 0.35 |
| Optiphen | 0.65 |
| Symdiol 68 | 0.60 |
| PART G | |
| Ethyl Alcohol SDA 40B - 190 Proof | 1.72 |
| Sorbic Acid NF | 0.15 |
| *TOTALS: | 100.00 |

### Procedure

1. For Part A combine the TEA and Daitosol. Mix together gently. Add the Water and Antifoam with very low stirring for 5 minutes. Adjust the pH level to 8.00-8.20 using TEA. Heat to 55° C. with stirring.
2. Add the NFCB-10D-2T in Part C to A. Disperse with the Cowles Mixer for 20 minutes
3. Add the pigment slurry Part D to AC and disperse with the Cowles mixer for 20 minutes.
4. Heat and slowly stir the Cetearyl Alcohol to 55° C. Add the Cetearyl Alcohol in Phase B to Part AC when both phases reach 55° C. Hold temperature for 10 minutes while mixing.
5. Begin slow cooling and add the PMMA and Micropoly 250S at 50° C. Continue dispersing with the Cowles mixer.
6. At 45° C. slurry together the ingredients of Part F and add to ABCDE.
7. Add the sorbic acid to the ethanol in PART G and stir together for 5 minutes. Add to the batch at 37° C.
8. Continue cooling and dispersing with the Cowles mixer while cooling to 27°-30° C. and fill.

### Example 5

### Thickening Mascara Concentrate (Low Heat Process)

| | % W/W |
|---|---|
| PART A | |
| Triethanolamine 99% | 0.29 |
| Daitosol 5000SJ | 24.03 |
| Antifoam C | 1.00 |
| PART B | |
| Cetearyl Alcohol and Ceteareth-20 | 8.85 |
| Micropoly ® 250S (polyethylene) | 5.00 |
| PART C | |
| MSS-500/N | 5.00 |
| PART D | |
| W60BBNFAP-O (Kobo Products) | 34.05 |
| PART E | |
| SUN PMMA-S | 18.25 |
| PART F | |
| Xanthan Gum | 0.35 |
| Optiphen | 0.65 |
| Symdiol 68 | 0.60 |
| PART G | |
| Ethyl Alcohol SDA 40B - 190 Proof | 1.78 |
| Sorbic Acid NF | 0.15 |
| *TOTALS: | 100.00 |

### Procedure

1. For Part A combine the TEA and Daitosol. Mix together gently. Add the Water and Antifoam with very low stirring for 5 minutes. Adjust the pH level to 8.00-8.20 using TEA. Heat to 55° C. with stirring.
2. Add the NFCB-10D-2T in Part C to A. Disperse with the Cowles Mixer for 20 minutes
3. Add the pigment slurry Part D to AC and disperse with the Cowles mixer for 20 minutes.
4. Heat and slowly stir the cetearyl alcohol to 55° C. Add the cetearyl alcohol in Phase B to Part AC when both phases reach 55° C. Hold temperature for 10 minutes while mixing.
5. Begin slow cooling and add the PMMA and Micropoly 250S at 50° C. Continue dispersing with the Cowles mixer.
6. At 45° C. slurry together the ingredients of Part F and add to ABCDE.
7. Add the sorbic acid to the ethanol in PART G and stir together for 5 minutes. Add to the batch at 37° C.
8. Continue cooling and dispersing with the Cowles mixer while cooling to 27°-30° C. and fill.

### Example 6

### Lash Primer or Topcoat Concentrate [Cold Process]

| | % W/W |
|---|---|
| Part A | |
| Triethanolamine 99% | 0.29 |
| Daitosol 5000SJ | 24.03 |
| (Acrylates/Ethylhexyl Acrylate Copolymer, Water) | |
| Deionized Water | 13.81 |
| Dow Corning ® Antifoam C | 1.00 |
| Part B | |
| Microcare ® 310 (polyethylene, Carnauba wax) | 4.75 |
| Micropoly ® 250S (polyethylene) | 9.00 |
| Part C | |
| SUN PMMA-S (PMMA, Sunjin) | 23.65 |
| MSS-500/N (Silica, Kobo) | 5.00 |
| Part D | |
| Sericite GMS-4C | 15.00 |
| Part E | |
| Xanthan Gum | 0.35 |
| Optiphen ® (Caprylyl Glycol, Phenoxyethanol) | 0.65 |
| Symdiol ® 68 (1,2-Hexanediol, Caprylyl Glycol) | 0.60 |
| Part F | |
| Ethyl Alcohol SDA 40B - 190 Proof | 1.72 |
| Sorbic Acid NF | 0.15 |
| Total: | 100.00 |

### Procedure

1. For Part A combine the TEA and Daitosol. Mix together gently. Add the Water and Antifoam with very low stirring for 5 minutes. Adjust the pH level to 8.00-8.20 using TEA.
2. Add the waxes in Phase B to Part A. Disperse with the Cowles Mixer at low speed for 20 minutes.
3. Add the Silica beads MSS-500/N in Part C and stir with the Cowles Mixer at low speed for 20 minutes.
4. Add the PMMA and stir with the Cowles Mixer at low speed for 20 minutes.
5. Add PART D Sericite to ABC. The viscosity will become thicker. Disperse with the Cowles Mixer at low speed for 20 minutes.
6. Slurry together the ingredients of PART E and add to Phase ABCD. Disperse very slowly with the Cowles Mixer at low speed for 20 minutes. Continue stirring until the air is reduced to a minimum.
7. Add the sorbic acid to the ethanol in PART E and stir together for 5 minutes. Add to the batch ABCDE. Disperse for 15 minutes.

The solids of the concentrates were tested by drying at 105 C for 2 h and the value was in a range of 70-75%. Viscosity was tested using a Brookfield viscosity RVT at 2.5 rpm and the results were a range of 150,000-350,000 cps. They are stable at room temperature and 45 C in 2 months stability test.

The concentrates can now be use to formulate a variety of the final mascara or eyeliner composition. The process is simple and easy. It requires only mixing at room temperature using a Cowles Mixer or the equivalent and a tank. No heating or homogenization is needed. The following are illustrative examples.

### Example 7

### Sepigel® 305 Mascara Formula from the Concentrate

| | % W/W |
|---|---|
| Part A | |
| Sepigel ® 305 (from Seppic) | 2.50 |
| (Polyacrylamide, C13-14 Isoparaffin, Laureth-7) | |
| Deionized Water | 47.00 |
| Optiphen ® Plus | 0.50 |
| Part B | |
| Lash Thickening Concentrate (Example 2) | 50.00 |

### Procedure:

1. Part A, begin mixing the deionized water in at room temperature then add the Sepigel 305 and stir for 20 minutes. Add the Optiphen Plus and stir for an additional 5 minutes.
2. Add the Lash Thickening Concentrate to the gel and stir for 20 minutes using a Cowles dissolver.
3. Fill.

### Example 8

### Natrosol® HHR CS Mascara formula from the concentrate

| | % W/W |
|---|---|
| Part A | |
| Natrosol ® HHR CS (From Aqualon) | 1.50 |
| (hydroxyethylcellulose) | |
| Deionized Water | 48.00 |
| Optiphen Plus | 0.50 |
| Part B | |
| Lash Lengthening Concentrate (Example 1) | 50.00 |

### Procedure:

1. Part A, begin mixing the deionized water at room temperature then add the Natrosol and stir for 45 minutes. Add the Optiphen Plus and stir for an additional 5 minutes.

2. Add the Lash Lengthening Concentrate to the gel and stir for 20 minutes using a Cowles dissolver.
3. Fill.

### Example 9

### Keltrol® CG Mascara Formula from the Concentrate

| | % W/W |
|---|---|
| Part A | |
| Keltrol ® CG (Xanthan Gum from CP Kelco) | 1.00 |
| Deionized Water | 48.50 |
| Optiphen ® Plus | 0.50 |
| Part B | |
| Lash Thickening Concentrate (Example 1) | 50.00 |

### Procedure:

1. Part A, begin mixing the deionized water at room temperature then add the Keltrol and stir for 45 minutes. Add the Optiphen Plus and stir for an additional 5 minutes.
2. Add the Lash Thickening Concentrate to the gel and stir for 20 minutes using a Cowles dissolver.
3. Fill.

### Example 10

### Keltrol® CG Eyeliner Formula from the Concentrate

| | % W/W: |
|---|---|
| Part A | |
| Keltrol ® CG (from CP Kelco) | 0.50 |
| Deionized Water | 49.00 |
| Optiphen ® Plus | 0.50 |
| Part B | |
| Eyeliner Concentrate (Example 3) | 50.00 |

### Procedure:

1. Part A, begin mixing the deionized water at room temperature then add the Keltrol and stir for 45 minutes. Add the Optiphen Plus and stir for an additional 5 minutes.
2. Add the Eyeliner Concentrate to the gel and stir for 20 minutes using a Cowles dissolver.
3. Fill.

### Example 11

### Silicone Eyeliner Formula from the Concentrate

| | % W/W: |
|---|---|
| Part A | |
| Dow Corning ® 5225C Formulation aid | 20.00 |
| Cyclopentasiloxane | 29.50 |
| Optiphen ® Plus | 0.50 |
| Part B | |
| Eyeliner Concentrate (Example 3) | 50.00 |

### Procedure:

1. Part A, begin mixing the two silicones in phase A at room temperature stirring for 10-15 minutes. Add the Optiphen Plus and stir for an additional 5 minutes.
2. Add the Eyeliner Concentrate to the silicone phase and stir for 20 minutes using a Cowles dissolver.
3. Fill.

### Example 12

### Silicone Mascara Formula from the Concentrate

| | % W/W: |
|---|---|
| Part A | |
| Dow Corning ® 5225C Formulation aid | 20.00 |
| Cyclopentasiloxane | 29.50 |
| Optiphen ® Plus | 0.50 |
| Part B | |
| Lash Thickening Concentrate (example 1) | 50.00 |

### Procedure:

1. Part A, begin mixing the two silicones in phase A at room temperature stirring for 10-15 minutes. Add the Optiphen Plus and stir for an additional 5 minutes.
2. Add the Lash Thickening Concentrate to the silicone phase and stir for 20 minutes using a Cowles dissolver.
3. Fill.

cl Example 13

### Sepigel® 305 Mascara Formula from the Concentrate

| | % W/W: |
|---|---|
| Part A | |
| Sepigel ® 305 (from Seppic) | 2.50 |
| Deionized Water | 44.50 |
| KTZ ® Roussillon | 2.50 |
| Optiphen ® Plus | 0.50 |
| Part B | |
| Lash Thickening Concentrate (Example 5) | 50.00 |

### Procedure:

1. Part A, begin mixing the deionized water in at room temperature then add the Sepigel 305 and stir for 20 minutes. Add the Optiphen Plus and stir for an additional 5 minutes.
2. Add the Lash Thickening Concentrate to the gel and stir for 20 minutes using a Cowles dissolver.
3. Fill.

### Example 14

### Natrosol® HHR CS Mascara Formula from the Concentrate

| | % W/W: |
|---|---|
| Part A | |
| Natrosol ® HHR CS (From Aqualon) | 1.50 |
| Deionized Water | 45.50 |
| KTZ Celandon Blue | 2.50 |
| Optiphen Plus | 0.50 |
| Part B | |
| Lash Lengthening Concentrate (Example 4) | 50.00 |

### Procedure:

1. Part A, begin mixing the deionized water at room temperature then add the Natrosol and stir for 45 minutes. Add the Optiphen Plus and stir for an additional 5 minutes.
2. Add the Lash Lengthening Concentrate to the gel and stir for 20 minutes using a Cowles dissolver.
3. Fill.

### Example 15

### Natrosol® HHR CS Lash Primer or Topcoat Formula from the Concentrate

| | % W/W: |
|---|---|
| Part A | |
| Natrosol ® HHR CS (From Aqualon) | 1.50 |
| Deionized Water | 48.00 |
| Optiphen Plus | 0.50 |
| Part B | |
| Lash Primer or Topcoat Concentrate (Example 6) | 50.00 |

### Procedure:

1. Part A, begin mixing the deionized water at room temperature then add the Natrosol and stir for 45 minutes. Add the Optiphen Plus and stir for an additional 5 minutes.
2. Add the Lash Lengthening Concentrate to the gel and stir for 20 minutes using a Cowles dissolver.
3. Fill.

### Comparison Studies

Leading marketed mascaras, 9 products from 8 brands, were purchased from Wal-Mart displays and used for the following tests:

### Rub Resistant Test

Rub resistance testing demonstrates the strength, adhesion and flexibility of the films incorporating the concentrate. Samples were weighed, applied to transpore tape to make films that were allowed to dry at 24 C for 24 hours. These films were abraded at a constant pressure and weight of 500 grams. The remaining film was digitized and transferred to image analysis software for results. A low score in the testing indicates by a low transfer during wear, also known as smudge-resistance (Table 1).

### Water Resistance Test

Water-resistance testing demonstrates the hydrophobic nature of the films incorporating the concentrate. A fixed amount of samples were weighed and applied in a Petri dish and allowed to dry at 24 C for 24 hours. The resultant films were then exposed to a circulating water bath for 4 hours. The remaining film was dried and weighed to determine its water-resistance. A high score in the testing indicating a high water-resistance during wear (Table 1).

### Color Analysis

For mascaras and eyeliners it is important that they have a deep, rich color or intensity. One gram of sample was applied and spread to a PMMA plate (3 inches in diameter) and allowed to dry for 24 hours. The color was analyzed by a Datacolor colorimeter using CIE Lab system. A lower L value indicates a darker color (Table 1).

TABLE 1
Test results

| | | Water | Rub- | |
|---|---|---|---|---|
| Commercial | | Resistance | Resistant | Darkness |
| mascara | Name | (%) | (%) | (L) |
| 1 | Maybelline Illegal | 82.79 | 5.53 | 15.95 |
| | Length | | | |
| 2 | L'Oreal Voluminous | 90.35 | 2.17 | 15.2 |
| | Naturale | | | |
| 3 | Maybelline Great Lash | 19.63 | 7.70 | 14.68 |
| 4 | Revlon Photoready | 68.68 | 3.84 | 17.42 |
| 5 | Wet N Wild | 79.23 | 8.14 | 16.85 |
| | Megaprotein | | | |
| 6 | NYC City Curls | 0.00 | 7.56 | 15.38 |
| 7 | Rimmel Volume | 80.16 | 3.22 | 14.46 |
| | Accelerator | | | |
| 8 | Revlon Grow Luscious | 10.01 | 2.96 | 19.14 |
| 9 | Neutrogena Healthy | 16.06 | 9.94 | 13.63 |
| | Volume | | | |
| Example 7 | | 95.35 | 0.06 | 9.72 |
| Example 8 | | 96.80 | 0.04 | 10.91 |
| Example 9 | | 96.62 | 0.07 | 11.00 |
| Example 12 | | 96.01 | 1.57 | 10.97 |

The foregoing examples and description of the preferred embodiments should be taken as illustrating, rather than as limiting the present invention as defined by the claims. As will be readily appreciated, numerous variations and combinations of the features set forth above can be utilized without departing from the present invention as set forth in the claims. All such variations are intended to be included within the scope of the following claims.

1. A dispersion composition comprising one or more waxes, one or more water-insoluble film-forming polymers, one or more pigments and other functional additives, wherein said dispersion has a viscosity in the range of about 150,000 cPs. to about 800,000 cPs. at about 25° C. and comprises at least about 60% of solids by weight.

2. The dispersion composition of claim 1, wherein said dispersion is an aqueous dispersion.

3. The dispersion composition of claim 1, wherein said one or more waxes comprise a plurality of wax particles.

4. The dispersion composition of claim 1, wherein said one or more waxes have particle sizes in the range of about 0.5 microns to about 50 microns (0.5-50 μm) in diameter.

5. The dispersion composition of claim 1, wherein said one or more waxes have particle sizes in the range of about 2 microns to about 10 microns (2-10 μm) in diameter.

6. The dispersion composition of claim 1, wherein said one or more waxes are independently selected from natural waxes.

7. The dispersion composition of claim 6, wherein said natural waxes comprise Carnauba wax, Candellila wax, Alfa wax, and Beeswax.

8. The dispersion composition of claim 1, wherein said one or more waxes are independently selected from synthetic waxes.

9. The dispersion composition of claim 8, wherein said synthetic waxes comprise polyethylene and microcrystalline waxes.

10. The dispersion composition of claim 1, wherein said one or more waxes comprise both synthetic and natural waxes.

11. The dispersion composition of claim 10, wherein said one or more waxes are present in the form of particles comprising a composite of synthetic and natural waxes.

12. The dispersion composition of claim 1, wherein particles of said one or more waxes are in the range from about 1% to about 30% by weight of the total composition.

13. The dispersion composition of claim 1, where said one or more water-insoluble film-forming polymers are emulsion of synthetic polymer emulsion or dispersion.

14. The dispersion composition of claim 1, wherein said one or more water-insoluble film-forming polymers are in the range of about 1% to about 30% by weight of the total composition.

15. The dispersion composition of claim 1, wherein said one or more water-insoluble film-forming polymers are independently selected from acrylate polymers or copolymers, polyurethanes, polyvinyl acetate and copolymers, styrene-butadiene copolymers, vinyl acetate-ethylene copolymers.

16. The dispersion composition of claim 1, wherein said one or more pigments are in the range of from about 2% to about 50% by weight of the total composition.

17. The dispersion composition of claim 1, wherein said one or more additive fillers comprise particulate materials.

18. The dispersion composition of claim 17, wherein said additive fillers are in a form selected from microspheres and fibers.

19. The dispersion composition of claim 18, wherein the microsphere has a size of about 1 μm to about 50 microns (1-50 μm) in diameter.

20. The dispersion composition of claim 18, wherein the microsphere is made from a mineral or polymeric material.

21. The dispersion composition of claim 18, wherein the fibers have a length in the range from about 0.1 mm to about 10 mm (0.1-10 mm) and a thickness in the range from about 0.5 denier to about 10 denier (0.5-10 D).

22. The dispersion composition of claim 18, wherein the fibers are made from a synthetic polymer or natural fibers.

23. The dispersion composition of claim 22, wherein said synthetic polymer is selected from Nylons.

24. The dispersion composition of claim 22, wherein said natural fiber is silk.

25. A method of making a dispersion composition of claim 1, comprising:
a) adjusting a polymer emulsion or dispersion with a base to a desired pH;
b) adding one or more waxes to the polymer emulsion or dispersion under agitation;
c) adding one or more functional additives to the mixture under agitation; and
d) adding one or more preservatives under agitation.

26. (canceled)

27. A method of making an eye composition, comprising:
a) mixing one or more diluents with one or more preservatives to form a mixture;
b) adding a dispersion composition according to claim 1 and mixing till uniform; and
c) filling with mixture obtained in b) with one or more filler materials.

28. The method of claim 27, wherein the diluents are selected from water, oil, silicone, and hydrocarbon fluids.

29. The method of claim 27, further adding a thickener to stabilize the final product.

30. (canceled)

31. (canceled)

32. (canceled)

33. (canceled)

34. A mascara incorporating the dispersion composition of claim 1 which is rub resistant, water resistant, or both water resistant and rub resistant.

35. The mascara of claim 34, having a dark color as measured by an L value in the range of 9 to 11.
